(19) United States
(12) Patent Application Publication
(10) Pub. No.: US 2021/0322600 A1
(43) Pub. Date: Oct. 21, 2021

(54) APPARATUS, SYSTEM AND METHOD FOR AUTOMATICALLY SANITIZING MACHINES

(71) Applicant: Shlomo Altman, Houston, TX (US)

(72) Inventor: Shlomo Altman, Houston, TX (US)

(21) Appl. No.: 17/235,834

(22) Filed: Apr. 20, 2021

Related U.S. Application Data
(60) Provisional application No. 63/012,919, filed on Apr. 21, 2020.

(51) Int. Cl. A61L 2/10; G06K 7/08; B08B 7/00; A61L 2/24

(52) U.S. Cl. CPC A61L 2/10; G06K 7/087; A61L 2202/14; A61L 2/24; B08B 7/0057

(57) ABSTRACT

This application describes an apparatus capable of automatically sanitizing commercial user interfaces including keypads of devices such as, a credit card payment machine between customers to prevent the transmission of bacteria, viruses, and other contagions that might otherwise be spread through contact with the devices. In addition, methods and systems are provided to sanitize devices.

### CROSS REFERENCE TO RELATED APPLICATIONS

The present application is a new United States Non-Provisional Patent Application that claims priority to co-pending U.S. Provisional Patent Application No. 63/012,919, entitled, “AN APPARATUS, SYSTEM AND METHOD FOR AUTOMATICALLY SANITIZING MACHINES, filed on Apr. 21, 2021, which is incorporated in its entirety by reference.

### FIELD

Embodiments of the invention relate to devices, methods, and systems that protect people from infectious diseases including pathogens such as, bacteria and viruses. More specifically, embodiments of the invention relate to devices, methods, and systems that protect users from infectious diseases using credit cards to pay for transactions. In one embodiment an Ultraviolet (UV) lamp and mounting apparatus is described for automatically sanitizing commercial keypads including credit card payment machine, automated teller machine (ATM), and gas pump credit card payment machine keypads.

### BACKGROUND

Preventing infectious diseases has become the biggest health need throughout the world. Infectious diseases are caused by microbes such as viruses and bacteria.

The coronavirus disease (COVID-19) is an infectious disease caused by a newly discovered coronavirus. Most people infected with the COVID-19 virus will experience mild to moderate respiratory illness and recover without requiring special treatment. Older people and those with underlying medical problems like cardiovascular disease, diabetes, chronic respiratory disease, and cancer are more likely to develop serious illnesses. The best way to prevent and slow down transmission is to be well informed about the COVID-19 virus, the disease it causes, and how it spreads and to protect yourself and others from infection by washing your hands or using an alcohol-based rub frequently and not touching your face.

The COVID-19 virus spreads primarily through droplets of saliva or discharge from the nose when an infected person coughs or sneezes. Therefore, people must practice respiratory etiquette (for example, by coughing into a flexed elbow).

While there are specific vaccines or treatments for COVID-19, the vaccines and treatments are not perfectly effective. Therefore, the public must reduce the likelihood of obtaining an infectious disease by maintaining social distancing, wearing masks, and sanitizing items and devices that are in contact with people in the public. Many businesses attempt to sanitize checkout areas using sanitizing sprays and manual cleaning between customers. Commercial keypads such as credit card payment machines are often overlooked in this process.

When cleaning of credit card payment machines is attempted, it is incomplete because sprays and wipes cannot reliably reach crevices between and around the keys on the keypads of these machines. It is also labor-intensive to clean between each customer. These commercial keypads include but are not limited to credit card payment machines, automated teller machines (ATM), and gas pump credit card payment machine keypads. Currently, these keypads are sanitized by spraying or wiping a disinfectant on the keypad. This time-consuming method is inefficient and ineffective as it is difficult, if not impossible, to constantly disinfect between customer use, and often the liquid can damage a keypad if too much is applied.

Therefore, society needs a better way to disinfect commercial keypads to protect the public from infectious diseases such as coronavirus. Accordingly, there is a need to provide automatic sanitization of commercial keypads that are efficient and effective without the risk of damaging the keypads. The embodiments of the present invention, as discussed below, satisfy these needs.

### SUMMARY

In one embodiment, the present disclosure is directed to an apparatus for sanitizing keypads. The apparatus comprises a slot for the payment card such as credit card or ATM card to be inserted into a chip reader; space for the user to access the keypad; at least one UV light; a mounting structure for mounting attachments to the UV light and an automatic shut-off switch to the apparatus; a power source; and a timer.

In a second embodiment, the present disclosure is further directed to a method of sanitizing devices. The method comprises several steps. First, a credit card is inserted into a reader. Second, a sensor detects the credit card. Third, at least one UV light directed toward the credit card reader is turned off while the credit card is detected. Fourth, the credit card is removed. Fifth, at least one UV light is turned on after a predetermined time after the card is no longer detected.

In a third embodiment, the present disclosure is further directed to a sanitizing system. The system comprises an apparatus with a slot for the credit card to be inserted into a chip reader; space for the user to access the keypad; at least one UV light; a sensor for detecting a credit card; a mounting structure for mounting attachments to the UV light and an automatic shut-off switch to the apparatus; a power source; a timer; and a control system for operating the UV light in a coordinated manner with the sensor and timer.

The foregoing is intended to give a general idea of the invention and is not intended to fully define nor limit the invention. The invention will be more fully understood and better appreciated by reference to the following description and drawings.

### BRIEF DESCRIPTION OF THE DRAWINGS

In the detailed description of various embodiments usable within the scope of the present disclosure, presented below, reference is made to the accompanying drawings, in which:

FIG. 1 illustrates an embodiment of the orientation of the apparatus concerning the machine, the positioning of the mounting attachments relative to the machine in this embodiment, the use of the payment card slot, and the possible location of a motion or proximity sensor used in an embodiment of the apparatus;

FIG. 2 illustrates an embodiment showing the UV light bulbs mounted to the underside of the top portion of the apparatus, a view of the mounting attachments, and the optional battery compartment;

FIG. 3 illustrates an automatic shut-off switch embodiment for the ultraviolet light;

FIG. 4 depicts an embodiment showing the apparatus installed on a credit card payment machine;

FIG. 5 describes a potential embodiment showing the steps of the apparatus' operation including how the UV light sanitation process works and how the apparatus responds to a user's input when a machine's chip-reader is used;

FIG. 6 describes the steps of one embodiment of an apparatus' operation embodiment showing how the UV light sanitation process works and how the apparatus responds to a user's input when a machine's magnetic strip reader is used; and

FIG. 7 describes the steps of one embodiment of the apparatus' operation including how the UV light sanitation process works and how the apparatus responds to a user's input when a motion sensor or proximity sensor is used to activate the UV light shut-off switch.

One or more embodiments are described below with reference to the listed Figures.

### DETAILED DESCRIPTION OF THE EMBODIMENTS

Before describing selected embodiments of the present disclosure in detail, it is to be understood that the present invention is not limited to the embodiments described herein. The disclosures and descriptions herein are illustrative and explanatory of one or more presently preferred embodiments and variations thereof, and it will be appreciated by those skilled in the art. Various changes in the design, organization, means of operation, structures and location, methodology, and use of mechanical equivalents may be made without departing from the spirit of the invention.

It should be understood that the drawings are intended to illustrate and disclose presently preferred embodiments to one of skill in the art, but are not intended to be manufacturing level drawings or renditions of final products and may include simplified conceptual views to facilitate understanding or explanation. The relative size and arrangement of the components may differ from that shown and still operate within the spirit of the invention.

Moreover, it will be understood that various directions such as “upper”, “lower”, “bottom”, “top”, “left”, “right”, “first”, “second” and so forth are made only with respect to explanation in conjunction with the drawings, and that components may be oriented differently, for instance, during transportation and manufacturing as well as operation. Because many varying and different embodiments may be made within the scope of the concepts herein taught, and because many modifications may be made in the embodiments described herein, it is to be understood that the details herein are to be interpreted as illustrative and non-limiting.

While businesses can require employees involved in sanitation to manually disinfect each credit card payment machine with a handheld UV light, this would be a labor-intensive sanitation solution subject to human error. In one embodiment, the apparatus described herein provides an automatic and thorough sanitation solution that is not available using strategically placed traditional ultraviolet lights.

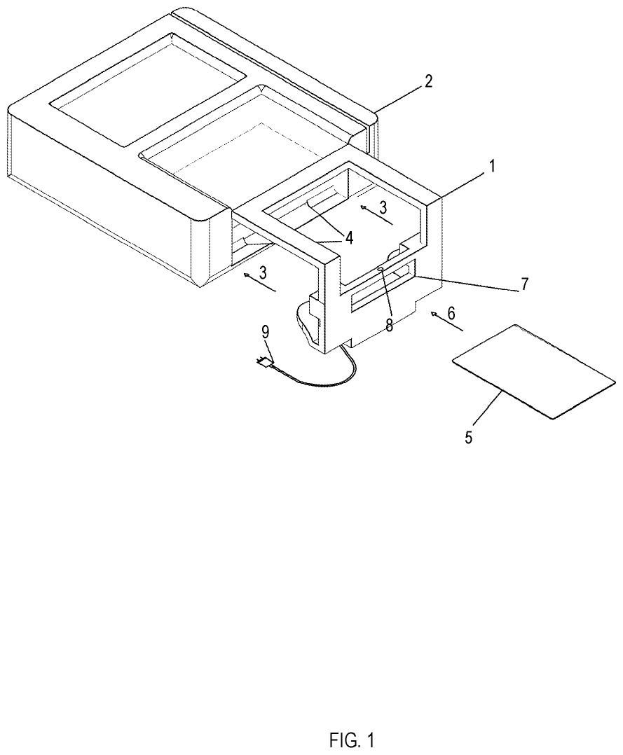

In one embodiment, the apparatus mounts onto a credit card payment machine. FIG. 1 illustrates the orientation of an embodiment of the apparatus structure 1, relative to a payment machine 2. The arrows 3 show the direction of attachment of the apparatus onto the machine and shows the position on the payment machine 4 where the apparatus mounting attachments would attach. A payment card is depicted 5 traveling in the direction shown by arrow 6 through the payment card slot 7. Above the card slot 8 is the likely placement of a motion or proximity sensor (not shown) described later. A plug 9 can be used to power the apparatus.

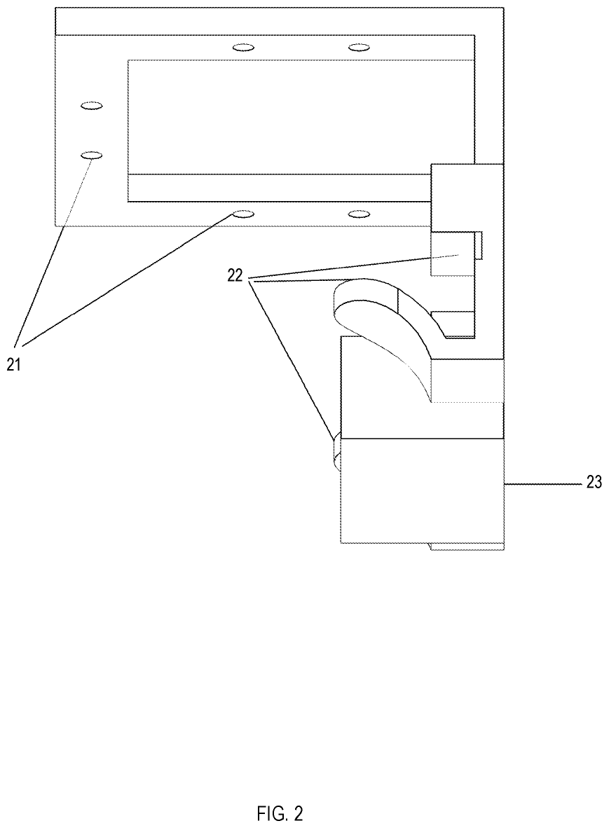

Now referring to FIG. 2, which illustrates an embodiment showing a plurality of light bulbs 21 mounted to the—underside of the upper portion of the apparatus. As shown in FIG. 2 the UV light bulbs 21, are positioned so the light shines down on the keypad. The apparatus mounting attachments 22 can be used to attach the apparatus to the payment machine. Preferably, there is space for a battery compartment 23 where one or more batteries would be placed and used to power an embodiment of the apparatus.

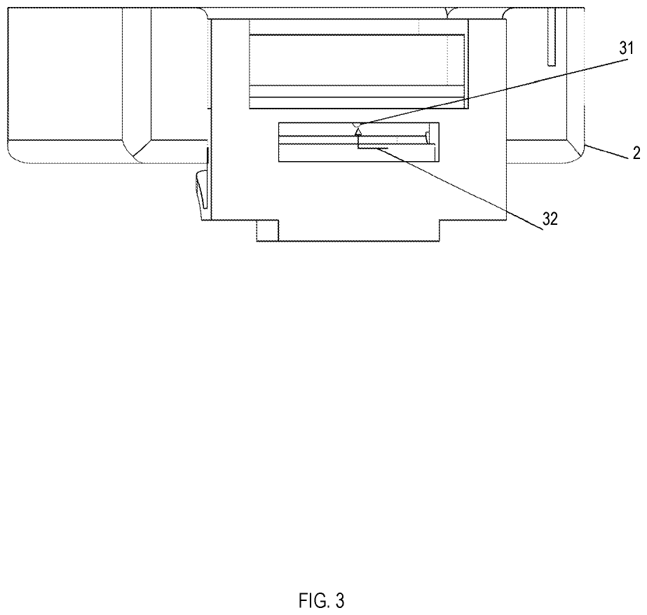

FIG. 3 illustrates the automatic shut-off switch 31 device or embodiment relative to a payment machine 2. Arrow 32 shows how credit card insertion depresses the button, shutting off the UV lights.

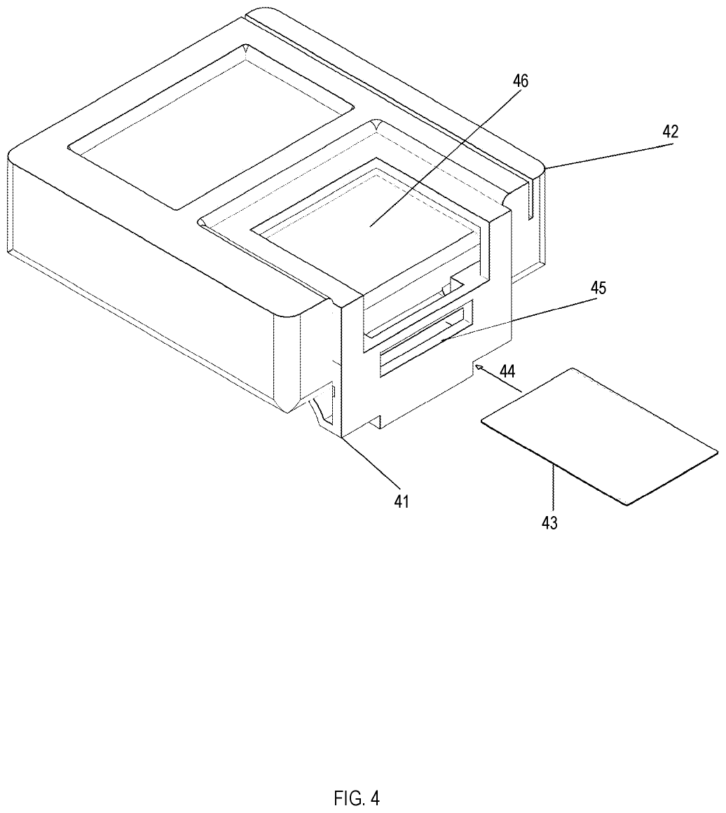

FIG. 4 shows apparatus 41 installed on a credit card payment machine 42. A payment card 43 is shown traveling in the direction of arrow 44 which is toward the payment card slot 45, included in apparatus 41. The UV lights, not shown in this figure, are installed in the underside of the upper portion of the apparatus and positioned over the keypad 46, to provide a seamless, minimally disruptive, and reliable method of sanitation.

UV light is an established method of sanitizing surfaces from viruses and bacteria. The wavelengths of light that are effective for sanitation purposes are UVC wavelengths, which are typically between 200 and 279 nm. See Rao, Bhamini K. et al. (2011). “Bactericidal Effect of Ultraviolet C (UVC), Direct and Filtered Through Transparent Plastic, on Gram-positive Cocci: An In Vitro Study,” Ostomy Wound Manage vol 57(7), 46-52. This reference is incorporated by reference in its entirety. More preferably the wavelengths are between 220 and 260 nm. The most used bactericidal wavelength is approximately 254 nm.

The bactericidal UVC (254 nm) dose required to inactivate different viruses is estimated at between 3 and 241 J/m2. See Kowalski, Wladyslaw & Walsh, Thomas & Petraitis, Vidmantas. (2020). 2020 COVID-19 Coronavirus Ultraviolet Susceptibility. 10.13140/RG.2.2.22803.22566 and Lytle, C. D., & Sagripanti, J. L. (2005). Predicted inactivation of viruses of relevance to biodefense by solar radiation. Journal of virology, 79(22), 14244-14252. https://doi.org/10.1128/JVI.79.22.14244-14252.2005. Both references are incorporated by reference in their entirety.

Various antibiotic-resistant bacterial strains required doses between 5 J/m2 and 12.5 J/m2 to be inactivated. See Sifuentes, Laura. (2015). Determination of Ultraviolet Light Doses Needed to Inactivate Bacteria and Viruses on Hard. Poster Abstracts, Presentation Number 2-115. American Journal of Infection Control, 43, S24. https://www.ncbi.nlm.nih.gov/pmc/articles/PMC3292282/. This reference is incorporated by reference in its entirety. Research suggests that viruses generally require higher doses for inactivation than bacteria. See id. Direct UVC (254 nm) eradicated 100% of multiple cultured bacterial strains over periods between 5 seconds and 90 seconds, at distances of approximately 1 in from the culture plates. See Dai, Tianhong et al. (2012). “Ultraviolet C irradiation: an alternative antimicrobial approach to localized infections?” Expert review of anti-infective therapy vol. 10(2), 185-95. doi:10.1586/eri.11.166. This reference is incorporated by reference in its entirety.

Direct exposure to a standard bactericidal UVC lamp, such as those used in biosafety cabinets, can kill the average bacteria in 10 seconds or less from an approximate distance of 6 inches. “Safety Tips for Using Germicidal Lamps,” Lawrence Berkely National Laboratory. https://lessonslearned.lbl.gov/Docs/2650/Germicidal%20lamp%20safety%20tips.pdf This reference is incorporated by reference in its entirety. According to a calculation provided by a major UV light manufacturer, at a distance of 1 ft from a 15 W bactericidal lamp (emitting light with 254 nm wavelength), a pathogen requiring 241 J/m2 of UVC exposure to be inactivated, would require 3.9 minutes of direct exposure for inactivation to be achieved. See Analytik Jena AG. https://www.uvp.com/products/ultraviolet-products/uv-handlamps/uvp-xx-series-uv-bench-lamps/. This reference is incorporated by reference in its entirety.

The 15 W lamp used in the calculation is an industry-standard, and 241 J/m2 represents a dose near the upper limit of what contagions might require to be inactivated. Considering these facts, and the short distance between the UV bulbs installed in the apparatus and the keypad, a required UV curing time of 5 minutes or less is likely to be achieved. The time to disinfect using a germicidal bulb depends on the following factors: the nature of the contagion such as, dose requirements for inactivation, the power (W) of the bulb, the frequency of UV light, and the distance from the light source to the surface of interest. Other factors that can affect dose requirements can include humidity and ambient temperature, though these factors have a greater impact over longer distances from the light source. Therefore, the UV light should be 12 inches or less from the keypad card reader or screen. The UV lightbulbs preferably should be a distance of at least one inch and less than 10 inches from the surface intended to be sanitized and more preferably less than 6 inches from the surface intended to be sanitized. Due to the frequency of customer use and depending on the exposure time as well as the intensity of the UV light, complete sanitation of the keypad would be difficult to be achieved for each customer if there is heavy use. However, in this case, the exposure of customers to contagion would still be dramatically reduced.

Depending on the wattage of the bulbs installed in the apparatus, more or less time may be required for complete sterilization. UV light cannot penetrate debris. Significant quantities of dirt or dust on the bulbs or keypad may inhibit the sanitation performance. In consideration of the unlikelihood that significant debris would be introduced to the keypads of credit card payment machines, it would likely be more than sufficient to wipe clean the apparatus, the keypad, and other exposed surfaces of the credit card payment machine once daily. Alternatively, an automatic cleaning solution can be provided with UV light to dissolve dirt and other debris. It is recommended that these areas be cleaned from dust and debris at least once per week. One easy and fast way to clean the keypad of credit card payment machines is with the use of compressed air, which can also be part of an automatic control system to maintain effective sanitation.

In an embodiment, the apparatus includes the following: UV lights 21, apparatus structure 1, mounting attachments 22, and an automatic shut-off switch 31. Configurations include a battery compartment 23, a power cable 9, or both. A timer (not shown) can be included inside the battery compartment. The apparatus includes a slot 7, for the payment card to be inserted into a chip reader, and space for the user to access the payment machine's keypad. Where an electrical outlet is not available, use of the apparatus' battery compartment is available or combinations can be considered, as needed.

The ability to favorably position UV lights around and above the user interface or keypad allows for thorough sanitizing between each row and column of keys. The UV bulbs used in the apparatus are placed in a mounting that surrounds the user interface or keypad of the credit card payment machine. The mounting is designed so the UV lights are typically at an elevated angle to the user interface or keypad, and so that multiple or polarity of UV bulbs are placed along the rear and sides of the user interface or keypad. The exact angle of elevation between the user interface or keypad and the mounting and the placement intervals of the bulbs within the mounting that is necessary to ensure direct light reaches all parts of the user interface or keypad varies between machine manufacturers and models. The installation and mounting of the apparatus, including the bulb mounting, will also vary between manufacturers and models. Therefore, it may or may not be possible to design a standardized model for most or all credit card payment machines, depending on how widely the dimensions vary between models and manufacturers.

Exposure to ultraviolet light can be harmful to users. To prevent overexposure of users to UV light, the apparatus includes a dome-shaped automatic shut-off switch that is activated when a customer inserts a credit card into the machine 31, and the credit card depresses the switch, as shown in FIG. 3. In this case, the card's presence in the payment slot physically depresses the shut-off switch, which is connected to a mechanism that turns off the UV lights, while the card remains inserted. As soon as the card is removed, the UV lights turn back on. Alternately, the shut-off switch may be triggered by a laser, motion detector or other non-contact sensor that can be used as a proximity sensor for the card and/or a user. In addition, other known proximity sensors can also be used. A timer (not shown) may be included to shut off the UV lights after a set time interval, when all viruses, bacteria, and other contagions have likely been eliminated. The timer may also be set to turn back on periodically to eliminate any contagion that may have contaminated the user interface or keypad without direct contact.

In another embodiment, the shut-off switch may be positioned above the card slot and in front of the user interface or keypad 8, as shown in FIG. 1. In this configuration the shut-off switch could be activated using a motion sensor or proximity sensor when a customer's hand approaches the user interface or keypad.

Some credit card payment machines use a touchscreen as well as a keypad. If the touchscreen positioning enables the apparatus to be attached above the touchscreen, additional ultraviolet lights can be added to the face toward the touchscreen. If necessary, the apparatus may also be built with an additional lamp structure to project the UV light onto the touchscreen. Most devices allow the user to answer touchscreen prompts via the keypad. If no lights are projecting UV light onto the touchscreen, then it is advisable to provide the user with options that allow the user to avoid contacting the touchscreen. For example, the user might be given the option to respond to touchscreen prompts using the keypad, or automatic payment processing may be enabled with card insertion.

Although the use of magnetic strip readers is a less common method of payment in these machines, a sensor that detects a card's passage through a magnetic card reader might be included that could provide an alternative mechanism for triggering the automatic shut-off switch. In this case, the use of a card in the magnetic card reader would trigger the UV lights to automatically shut off, reducing the user's UV exposure. This could be accomplished by extending the structure in front of the magnetic reader with a slot for the card to pass through. This slot would be equipped with a sensor that detects the presence of a card. This sensor could be a physical switch, comparable to the one shown in FIG. 3 as element 31, or it could be a laser sensor, motion detector, or other non-contact sensors. When the card passes through the slot and the card is detected, the shut-off switch is activated. Once a card is detected by the sensor attached to the magnetic card reader, and the UV lights are turned off, the UV lights remain off for a preprogrammed time interval. This time interval is chosen to allow the user sufficient time to authorize payment and enter the pin number. Once the internal timer indicates the programmed interval is passed, an internal mechanism to turn on the UV lights is activated.

UVC light does not penetrate as deeply into human skin as other wavelengths of UV light. However, maximum safe daily dosages of light at these wavelengths are not well-established, and corneal burns have occurred from intense and prolonged exposures to these wavelengths. Therefore, it is recommended that a UV-resistant shield be included over the apparatus.

The most effective UV shield would cover all exposed areas of the apparatus. One possible embodiment of an apparatus with an integrated UV shield is to form the UV shield as a hinged cover with a latch that secures the cover to the mounting of the apparatus. The cover would be large enough to cover the apparatus as well as the entire area of the credit card payment machine to be sanitized. In this configuration, an automatic shut-off switch could be triggered when the latch is released, and the cover is lifted. Then, when the cover is put down, a switch to turn on the UV lights would be triggered. Additionally, to reduce energy expenditures and extend the life of UV bulbs, a timer could be installed to shut off the UV lights after a predetermined time interval passes. This time interval would be calculated with the following variables: the maximum distance between a surface to be sanitized and the bulbs, the wattage of the UV bulbs, and the maximum dose requirements of relevant pathogens.

The use of a UV shield would also prevent dust and particulates from settling onto the keypads and other sanitized surfaces. The use of the UV shield is recommended to ensure customer safety. Each customer would lift the cover to the open position, complete their transaction, and then close the cover for the next customer.

For gas station pumps and automatic teller machines (ATM) a similar structure is utilized, with the exclusion of the mount attachments. Alternatively, the mount may be attached using other means, such as an adhesive or wrap, around the structure mount.

Alternatively, the apparatus or device can be a UV light that can operate independently or be integrated into the design of a machine and manufactured as one unit, with the card detection, and off switch for the UV lamp integrated into the card reader and the UV lights built into the machine. In one embodiment, that apparatus is a UV lamp that sanitizes a user interface: allowing observation of, and interaction with, the interface while simultaneously preventing a user from direct UV light exposure to the skin and eyes. The UV lamp allows the user to seamlessly interact with the user interface and is automatically deactivated when the user begins to interact with the user interface. This occurs before the user contacts the area being sanitized preventing the UV light from contacting the user's body including the skin or eyes.

### Method:

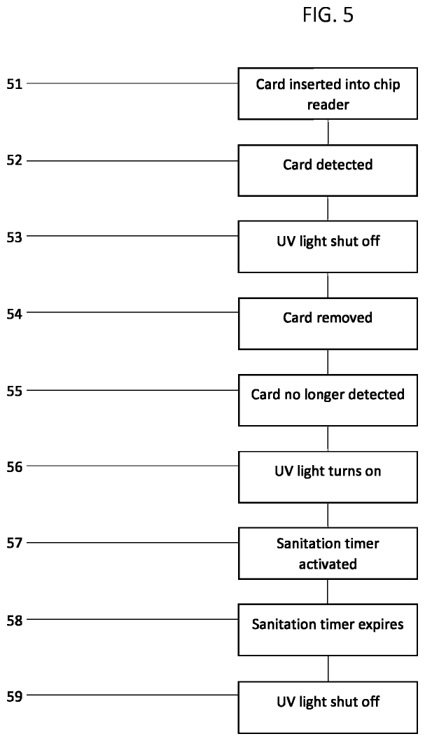

FIG. 5 describes a potential embodiment showing the steps of the apparatus' operation including how the UV light sanitation process works and how the apparatus responds to a user's input when a machine's chip-reader is used. When a card is inserted for payment into the chip reader 51, a card is detected 52, and the UV light shut-off switch is activated 53. When the user has finished inputting the necessary commands, authorization, and/or, PIN, the user removes card 54. The system no longer detects the payment card 55, which triggers a switch to turn the UV light on 56. The UV lights in the apparatus then begin to sanitize the keypad surface. An internal timer is activated 57, and the lights remain on until the timer expires 58. The expiration of the timer triggers the UV lights to turn off 59. The timer is set for a time that is estimated to sufficiently sanitize the keypad, as discussed above. The internal timer may also be set to periodically turn on the UV lights automatically to sanitize the keypad from airborne contaminants that may have fallen onto the keypad surface. If a card is detected before method Step 58 is complete, or at any other point, the system would reset to method step 51.

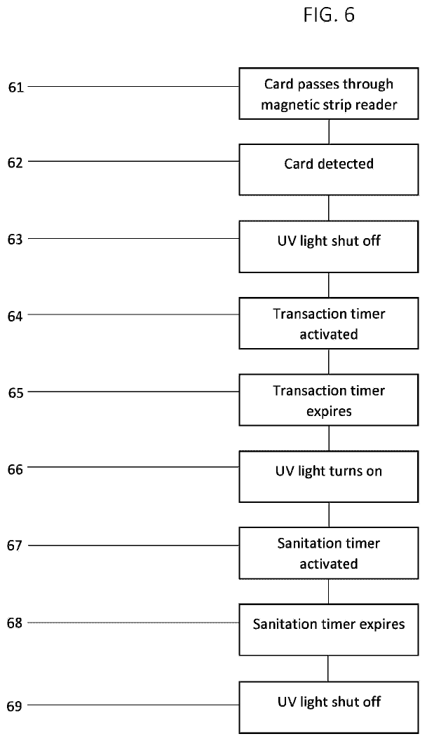

FIG. 6 describes the steps of an apparatus' operation embodiment showing how the UV light sanitation process works and how the apparatus responds to a user's input when a machine's magnetic strip reader is used. When a card is detected exiting a magnetic strip reader. First, the card passes through reader 61 and is detected 62. Second, a UV shut-off switch is activated 63. Third, an internal timer, set to allow the user sufficient time to input the necessary commands, authorization, and/or, PIN number, is activated 64. Fourth, when the timer expires 65, the UV light is turned on to begin sanitation 65. Another internal timer is activated 67 and the lights remain on until the timer expires 68. The expiration of the timer triggers the UV lights to turn off 69. The timer is set for a time that is estimated to be sufficient to sanitize the keypad. The internal timer may also be set to periodically turn on the UV lights automatically to sanitize the keypad from airborne contaminants that may have fallen onto the keypad surface. If a card (and therefore a user) is detected before step 8. is complete, or at any other point, the system can selectively close the machine or reset to the first step 61.

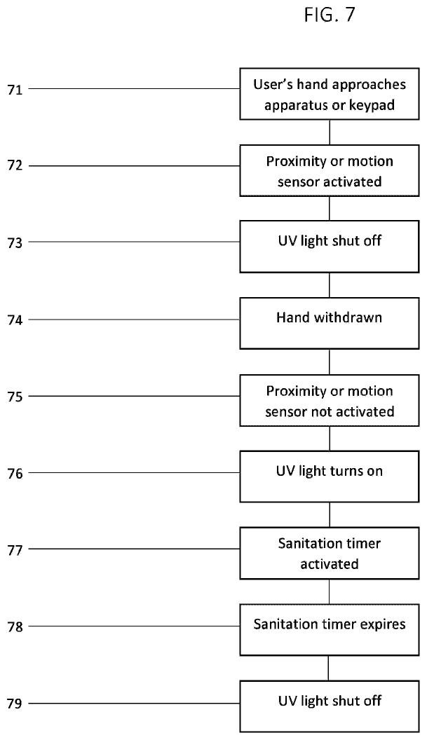

FIG. 7 describes the steps of the apparatus' operation including how the UV light sanitation process works and how the apparatus responds to a user's input when a motion sensor or proximity sensor is used to activate the UV light shut-off switch. When a hand approaches the keypad or apparatus 71, motion or an interruption in the proximity sensors range is detected 72. Activation of the motion or proximity sensor triggers the UV light to turn off 73. When the user has completed the transaction and withdrawn their hand 74, the motion or proximity sensors no longer detect movement or interruption 75, the UV light is turned on 76. An internal timer is activated 77 and the lights remain on until the timer expires 78. The expiration of the timer triggers the UV lights to turn off 79. The timer is set for a time that is estimated as sufficient to sanitize the keypad. The internal timer may also be set to periodically turn on the UV lights automatically to sanitize the keypad from airborne contaminants that may have fallen onto the keypad surface. If a card (and therefore a user) is detected before method step 78 is complete, or at any other point, the system can selectively close the machine or reset to method step 71.

### System:

In an embodiment of the apparatus, a control system is included to operate the UV light in a coordinated manner with the sensor and timer. The control system can either be an electrical control system, a computerized control system, or a SCADA system. The control system can be comprised of nested loops, cascades, or separate modular control loops. The control protocols described herein are not intended to be limiting, but to provide examples of possible control systems.

A combined feedforward and feedback control system can control the operation of the apparatus. The user's interaction with a physical or non-contact sensor provides an input to the control system. An output of the control system is the operation of the UV lights. Timer(s) can be installed in embodiments. The activation of timer(s) function as additional input(s) and output(s) of the control system. The expiration of timer(s) function as additional input(s) to the control system.

When a user first interacts with the sensor, the user input protocol is initiated by the control system. A description of the user input protocol follows. The control system turns the UV lights off. At this point, depending on the embodiment, the control system performs one of two sets of actions. First, the control system continuously detects user interaction via the physical or non-contact sensor. In response, the control system keeps the UV light(s) off In this embodiment, when the control system detects that user interaction has ceased, via the sensor, the control system turns the UV light(s) on.

Alternatively, the control system activates a timer and keeps the UV light(s) off until the timer expires. This timer can either be factory programmed by the manufacturer or programmed by the owner of the payment machine. The timer is set to allow enough time for the customer to complete their transaction. In this embodiment, when the timer expires, the control system detects the input from the timer and turns the UV light(s) on. This ends the user input protocol.

Following the user input protocol, the sanitation protocol is initiated. A description of the sanitation protocol follows. After the UV light(s) have been turned on, the control system activates a timer. This timer can either be factory programmed by the manufacturer or programmed by the owner of the payment machine. The timer is set to allow enough time for the UV light(s) to sanitize the keypad (or any other surface intended to be sanitized by the apparatus) In this embodiment when the timer expires, the control system detects the input from the timer and turns the UV light(s) off.

Following the completion of the sanitation protocol, an embodiment is programmed to follow a standby protocol. A description of the standby protocol follows. In an embodiment, the apparatus can be programmed with an additional timer. This timer can either be factory programmed by the manufacturer or programmed by the owner of the payment machine.

Following the sanitation protocol, the control system activates the timer. This timer is set with a defined time, after which, in the absence of user contact, the timer would expire. When this timer expires, the control system would turn on the UV light(s) and initiate the sanitation protocol. If the sanitation protocol or the standby protocol is interrupted by user input, the control system overrides these protocols and initiates the user input protocol. It should also be understood that each feature described above can be incorporated into many types of commercial and residential devices, without departing from the scope of the present disclosure.

What is claimed is:

1. An apparatus comprising:
a. a slot for the credit card to be inserted into a chip reader;
b. space for the user to access the keypad;
c. at least one UV light;
d. a mounting structure and mounting attachments for mounting the at least one UV light to a payment machine and an automatic shut-off switch to the apparatus;
e. a power source; and
f. a timer.

2. The apparatus of claim 1 wherein the power source is a plug, battery, and combinations thereof.

3. The apparatus of claim 1, further comprising additional UV lights wherein a plurality of UV lights are at an elevated angle with respect to the keypad.

4. The apparatus of claim 3 wherein a first plurality of UV bulbs are placed along a rear section of the keypad and a second plurality of UV bulbs are placed on the sides of the keypad.

5. The apparatus of claim 1 wherein additional UV bulbs are projected onto a screen adjacent to the reader.

6. The apparatus of claim 5 wherein the screen is a touchscreen.

7. The apparatus of claim 1 further comprising a UV shield.

8. The apparatus of claim 1 further comprising a sensor attached adjacent to the magnetic card reader for turning off the UV lights when a credit card is inserted into the reader or is adjacent to the reader and an internal mechanism to turn on the UV lights after a predetermined time set with the timer.

9. The apparatus of claim 1 wherein the apparatus is chosen from the group consisting of credit card machine, a gas station pump, automatic teller machines, and combinations thereof.

10. The apparatus of claim 1 further comprising a control system to operate the apparatus and UV light in a coordinated manner.

11. A method comprising:
a. inserting a credit card into a reader;
b. detecting the credit card;
c. turning off at least one UV light directed toward the credit card reader while the credit card is detected;
d. removing the credit card; and
e. turning on the at least one UV light after a predetermined time after the card is no longer detected.

12. The method of claim 11 further comprising using a UV light directed to a keypad that is turned off when a credit card is detected.

13. The method of claim 11 further comprising shutting off the UV light after a predetermined time even if no credit card or user is detected.

14. A system comprising:
a. an apparatus with a slot for the credit card to be inserted into a chip reader;
b. space for the user to access a user interface;
c. at least one UV light;
d. a sensor for detecting a credit card;
e. a mounting structure for mounting attachments to the at least one UV light and an automatic shut-off switch to the apparatus;
f. a power source;
g. a timer; and
h. a control system for operating the at least one UV light in a coordinated manner with the sensor and timer.

15. The system of claim 14, further comprising additional UV lights wherein a plurality of UV lights are at an elevated angle with respect to the user interface, wherein the user interface is a keypad.

16. The system of claim 14, further comprising wherein a first plurality of UV lights are placed along a rear section of the keypad and a second plurality of UV lights are placed on the sides of the user interface.

17. The system of claim 14, further comprising wherein additional UV bulbs are projected onto a screen adjacent to the reader.

18. The system of claim 14, further comprising further comprising a UV shield that can be controlled by the control system.

19. The system of claim 14, wherein the control system keeps the UV light off long enough and disables the credit card reader to allow sufficient time for microbes, viruses, bacteria, pathogens and combinations thereof to be killed.

20. The system of claim 14 wherein the at least one UV light that sanitizes the user interface; allows the user interaction with the interface by automatically deactivating when the user begins to interact with the user interface, and wherein deactivation occurs before the user makes contact with the area being sanitized to prevent the UV light from contacting the user's body.
